(19) United States
(12) Patent Application Publication
(10) Pub. No.: US 2025/0009204 A1
(43) Pub. Date: Jan. 9, 2025

(54) STATE DETERMINATION METHOD FOR ENDOSCOPE PIPE LINE, STATE DETERMINATION DEVICE FOR ENDOSCOPE PIPE LINE, AND ENDOSCOPE WASHING AND DISINFECTION DEVICE

(71) Applicant: FUJIFILM Corporation, Tokyo (JP)

(72) Inventors: Takashi HARADA, Kanagawa (JP); Kimitake FUKUSHIMA, Kanagawa (JP)

(73) Assignee: FUJIFILM Corporation, Tokyo (JP)

(21) Appl. No.: 18/889,335

(22) Filed: Sep. 18, 2024

Related U.S. Application Data
(63) Continuation of application No. PCT/JP2023/009248, filed on Mar. 10, 2023.

(30) Foreign Application Priority Data
Mar. 22, 2022 (JP) 2022-045702

(51) Int. Cl. A61B 1/00; A61B 1/015

(52) U.S. Cl. CPC A61B 1/00057; A61B 1/00006; A61B 1/015

(57) ABSTRACT

Provided are a state determination method for an endoscope pipe line, a state determination device for an endoscope pipe line, and an endoscope washing and disinfection device with which it is possible to determine whether an endoscope pipe line is in an open or blocked state with high accuracy. A state determination method for an endoscope pipe line includes a supply step of supplying a pressurized fluid into an endoscope pipe line, a change rate acquisition step of acquiring a change rate of a physical quantity of the fluid within a determination period after the supply of the fluid is stopped, and a determination step of determining whether the endoscope pipe line is in an open or blocked state based on the change rate acquired in the change rate acquisition step.

### CROSS-REFERENCE TO RELATED APPLICATIONS

The present application is a Continuation of PCT International Application No. PCT/JP2023/009248 filed on Mar. 10, 2023 claiming priority under 35 U.S.C § 119 (a) to Japanese Patent Application No. 2022-045702 filed on Mar. 22, 2022. Each of the above applications is hereby expressly incorporated by reference, in its entirety, into the present application.

### BACKGROUND OF THE INVENTION

### 1. Field of the Invention

The present invention relates to a state determination method for an endoscope pipe line, a state determination device for an endoscope pipe line, and an endoscope washing and disinfection device, and particularly to a state determination method for an endoscope pipe line, a state determination device for an endoscope pipe line, and an endoscope washing and disinfection device for determining whether an endoscope pipe line is in an open or blocked state in a washing treatment of an endoscope.

### 2. Description of the Related Art

An endoscope used in a medical field is used by being inserted into a body cavity for the purpose of an examination and a treatment. Therefore, after use, it is necessary to perform washing and disinfection in order to use the endoscope again. As a device for washing and disinfecting the used endoscope, an endoscope washing and disinfection device is known. The endoscope washing and disinfection device usually performs washing and disinfection through a plurality of steps such as washing, disinfection, and rinsing.

In this case, the washing and the disinfection are also performed on a plurality of endoscope pipe lines in the endoscope, such as an air/water supply pipe line, a suction pipe line, and a treatment tool insertion pipe line, by supplying a washing solution and a disinfectant solution into the endoscope. In such a case, in a case in which a blockage (clogging) of the pipe line of the endoscope has occurred, it is difficult for the washing solution and the disinfectant solution to be supplied into the pipe line, so that sufficient washing and disinfection are not performed. In addition, during an operation of the endoscope after the washing, the supply and the suction of a fluid or the like cannot be performed through the pipe line. Therefore, a test for determining whether the pipe line of the endoscope is in an open or blocked state is performed.

For example, WO2004/049925A describes that a clogged state of an endoscope is detected by supplying a fluid into a pipe line of the endoscope, measuring a pressure or a flow rate of the fluid flowing through the pipe line, and performing a comparison operation between the measured value and a set value. JP2009-514611A describes that whether a channel is connected and open or not connected is determined by sending a pressurized fluid to a channel in an endoscope, monitoring a back pressure, and monitoring a time for the back pressure to drop to a predetermined value.

JP2011-521751A describes that the series of pressure pulses are applied to an endoscope pipe line, and an opening property of the endoscope pipe line is tested based on a maximum value and a minimum value of the pressure pulses. JP2006-230709A describes that an abnormality is determined by supplying a fluid into an internal pipe line of an endoscope, and comparing a pressure or a flow rate of the fluid with a threshold value.

### SUMMARY OF THE INVENTION

In a case of determining whether the pipe line of the endoscope is in the open or blocked state, as described in WO2004/049925A, JP2009-514611A, JP2011-521751A, and JP2006-230709A, the state of the pipe line of the endoscope is determined based on the value of the pressure or the flow rate of the fluid supplied into the pipe line of the endoscope, or the time required for the pressure or the flow rate to be the predetermined value.

However, in a case in which the pressure or the flow rate of the fluid is measured, variation may occur in the value of the pressure or the flow rate due to the following factors. (1) An attenuation waveform has a change point, but a position of the change point is different depending on a type of the endoscope or the open state of the pipe line. (2) The waveform during the attenuation is disturbed due to a variation in a measurement device, an influence of pipe line turbulence, or the like. (3) An outlier is generated due to an influence of noise or the like. Therefore, in a case in which a variation occurs in a value of the pressure or the flow rate of the fluid, there is a possibility that an erroneous determination will occur.

The present invention has been made in view of such circumstances, and an object of the present invention is to provide a state determination method for an endoscope pipe line, a state determination device for an endoscope pipe line, and an endoscope washing and disinfection device with which it is possible to determine whether an endoscope pipe line is in an open or blocked state with high accuracy.

A first aspect relates to a state determination method for an endoscope pipe line, comprising: a supply step of supplying a pressurized fluid into an endoscope pipe line; a change rate acquisition step of acquiring a change rate, which is a change amount per unit time of a physical quantity of the fluid within a determination period after the supply of the fluid is stopped; and a determination step of determining whether the endoscope pipe line is in an open or blocked state based on the change rate acquired in the change rate acquisition step.

A second aspect relates to the state determination method for an endoscope pipe line, in which the physical quantity is a pressure or a flow rate of the fluid.

A third aspect relates to the state determination method for an endoscope pipe line, in which the supply of the fluid is stopped after the endoscope pipe line is filled with the fluid in the supply step.

A fourth aspect relates to the state determination method for an endoscope pipe line, in which the change rate acquisition step includes a detection step of detecting physical quantity data indicating a physical quantity of the fluid corresponding to each of a plurality of time points within the determination period, and a calculation step of calculating the change rate based on the physical quantity data detected in the detection step.

A fifth aspect relates to the state determination method for an endoscope pipe line, in which the calculation step includes conversion processing of converting the change rate into a constant.

A sixth aspect relates to the state determination method for an endoscope pipe line, in which, in the calculation step, as the conversion processing, logarithmic conversion is performed on at least one of the physical quantity data or time data indicating an elapsed time from a start of the determination period, to convert the change rate into the constant.

A seventh aspect relates to the state determination method for an endoscope pipe line, in which, in the calculation step, the change rate is calculated based on time-division data obtained by time-dividing the physical quantity data for each time.

An eighth aspect relates to the state determination method for an endoscope pipe line, in which, in the calculation step, the change rate is calculated by performing linear approximation of the time-division data.

A ninth aspect relates to the state determination method for an endoscope pipe line, in which, in the calculation step, the change rate is calculated based on a slope between two points included in the time-division data.

A tenth aspect relates to the state determination method for an endoscope pipe line, in which, in the calculation step, the change rate is calculated by performing linear approximation based on a residual of the time-division data.

An eleventh aspect relates to the state determination method for an endoscope pipe line, in which, in the calculation step, the change rate is calculated by performing linear approximation in which a sum of squares of residuals of the time-division data is minimized.

A twelfth aspect relates to the state determination method for an endoscope pipe line, further comprising: an outlier exclusion step of specifying an outlier included in the physical quantity data based on the physical quantity data after the conversion processing is performed, to exclude the outlier from the physical quantity data.

A thirteenth aspect relates to the state determination method for an endoscope pipe line, further comprising: a variation determination step of determining a degree of a variation in the physical quantity data based on the physical quantity data after the conversion processing is performed.

A fourteenth aspect relates to the state determination method for an endoscope pipe line, in which, in the determination step, the determination is performed by comparing the change rate that is converted into the constant by the conversion processing with a determination threshold value indicating whether the endoscope pipe line is open or blocked.

A fifteenth aspect relates to the state determination method for an endoscope pipe line, in which the determination period is a period after a preset exclusion period has elapsed from the stop of the supply of the fluid.

A sixteenth aspect relates to a state determination device for an endoscope pipe line, comprising: a supply pipe line that is connected to an endoscope pipe line and that supplies a pressurized fluid into the endoscope pipe line; a physical quantity detection sensor that detects a physical quantity of the fluid; and a processor, in which the processor acquires a change rate, which is a change amount per unit time of the physical quantity of the fluid within a determination period after the supply of the fluid is stopped, based on the physical quantity of the fluid detected by the physical quantity detection sensor, and determines a state of the endoscope pipe line based on the calculated change rate.

A seventeenth aspect relates to the state determination device for an endoscope pipe line, in which the physical quantity is a pressure or a flow rate of the fluid.

An eighteenth aspect relates to the state determination device for an endoscope pipe line, in which the supply of the fluid is stopped after the endoscope pipe line is filled with the fluid.

A nineteenth aspect relates to the state determination device for an endoscope pipe line, in which the processor detects physical quantity data indicating a physical quantity of the fluid corresponding to each of a plurality of time points within the determination period, and calculates the change rate based on the detected physical quantity data.

A twentieth aspect relates to the state determination device for an endoscope pipe line, in which the processor performs conversion processing of converting the change rate into a constant.

A twenty-first aspect relates to an endoscope washing and disinfection device comprising: the state determination device for an endoscope pipe line described above.

According to the aspects of the present invention, it is possible to determine whether the endoscope pipe line is in the open or blocked state with high accuracy.

### BRIEF DESCRIPTION OF THE DRAWINGS

FIG. 1 is an overall view of an endoscope to be washed by an endoscope washing device according to an embodiment.

FIG. 2 is a main part perspective view showing a distal end side of an insertion part of the endoscope.

FIG. 3 is a schematic configuration diagram of the washing device.

FIG. 4 is a block diagram of a controller.

FIG. 5 shows a connection configuration between a state determination device and an endoscope pipe line of an endoscope.

FIG. 6 is a flowchart of a state determination method for an endoscope pipe line.

FIG. 7 is a flowchart of a supply step.

FIG. 8 is a graph showing a change in a pressure in a supply pipe line in the supply step shown in FIG. 7.

FIG. 9 is a graph of an actual pressure value for a determination period of FIG. 8.

FIG. 10 is a flowchart of a change rate acquisition step.

FIG. 11 is a semi-logarithmic graph in which a horizontal axis is a time axis and a vertical axis is a logarithmic axis of the pressure.

FIG. 12 is a graph obtained by performing conversion processing on the graph shown in FIG. 11 such that a change rate is converted into a constant.

FIG. 13 is a flowchart of a determination step.

FIG. 14 is a diagram showing a comparison between the change rate and a threshold value.

### DESCRIPTION OF THE PREFERRED EMBODIMENTS

Hereinafter, a state determination method for an endoscope pipe line, a state determination device for an endoscope pipe line, and an endoscope washing and disinfection device according to preferred embodiments of the present invention will be described with reference to the accompanying drawings.

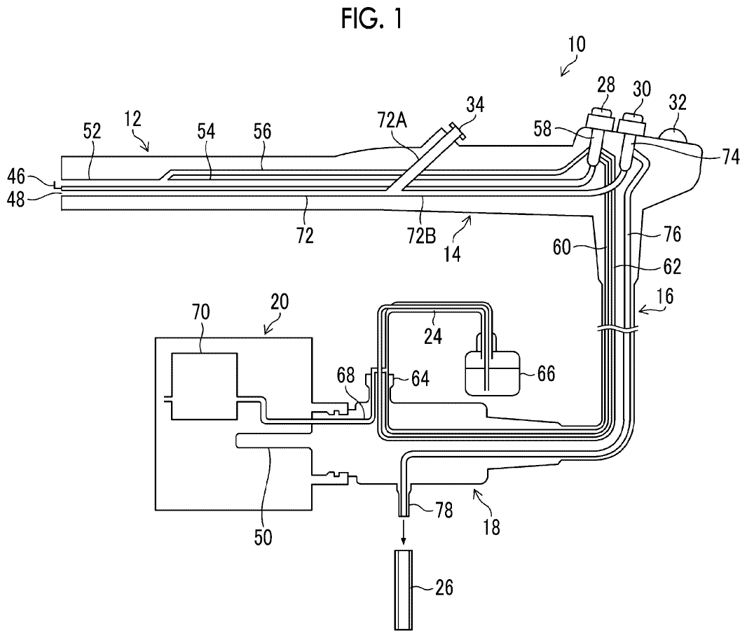

FIG. 1 is an overall view of an endoscope 10 of which a state of a pipe line is determined by a state determination method for an endoscope pipe line according to the embodiment, and particularly schematically shows a pipe line configuration of the endoscope 10. First, a configuration of the endoscope 10 will be described with reference to FIG. 1.

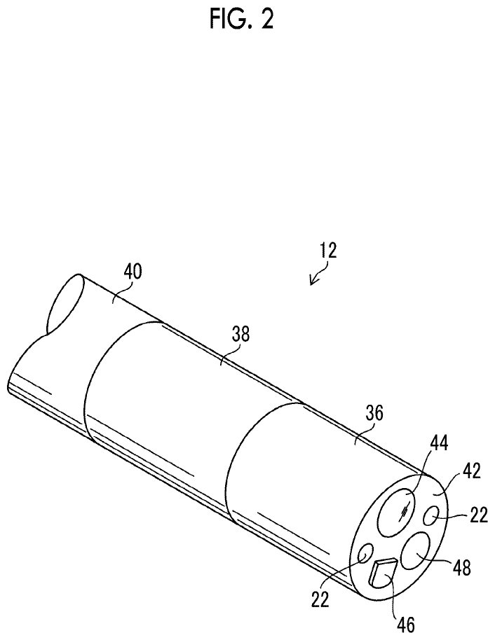

As shown in FIG. 1, the endoscope 10 comprises an insertion part 12 to be inserted into a lumen of a patient, for example, into a digestive tract such as a stomach or a large intestine, and a hand-side operating part 14 that is installed consecutively with the insertion part 12. A universal cable 16 is connected to the hand-side operating part 14, and an LG connector 18 is provided at a distal end of the universal cable 16. By connecting the LG connector 18 to a light source device 20, illumination light is transmitted to illumination windows 22 and 22 (see FIG. 2). In addition, the LG connector 18 includes an electrical connector (not shown), and the electrical connector is attachably and detachably connected to a processor (not shown). It should be noted that a pipe line 24 for air/water supply and a pipe line 26 for suction are connected to the LG connector 18.

An air/water supply button 28, a suction button 30, and a shutter button 32 are installed adjacent to the hand-side operating part 14, and a pair of angle knobs (not shown) and a forceps insertion port 34 are provided in the hand-side operating part 14.

FIG. 2 is a main part perspective view showing a distal end side of the insertion part 12. As shown in FIG. 2, the insertion part 12 has a distal end part 36, a bendable part 38, and a soft part 40, and the bendable part 38 is bent remotely by rotationally moving the angle knobs provided in the hand-side operating part 14. Accordingly, a distal end surface 42 of the distal end part 36 can be directed in a desired direction.

An observation window 44, the illumination windows 22 and 22, an air/water supply nozzle 46, and a forceps port 48 are provided on the distal end surface 42 of the distal end part 36. An imaging element (not shown) is disposed behind (base end side) the observation window 44. The imaging element is supported by a substrate (not shown), and a signal cable is connected to the substrate. The signal cable is inserted into the insertion part 12, the hand-side operating part 14, and the universal cable 16 of FIG. 1, is projected to the electrical connector, and is connected to the processor. Therefore, an observation image taken in from the observation window 44 of FIG. 2 is formed on a light-receiving surface of the imaging element and converted into an electrical signal, and this electrical signal is output to the processor via the signal cable and converted into a video signal. As a result, the observation image is displayed on a monitor (not shown) connected to the processor. It should be noted that a charge coupled device (CCD) image sensor or a complementary metal oxide semiconductor (CMOS) image sensor is used as the imaging element.

An emission end of a light guide (not shown) is disposed behind (base end side) the illumination windows 22 and 22. The light guide is inserted into the insertion part 12, the hand-side operating part 14, and the universal cable 16 of FIG. 1. Then, the incidence end of the light guide is connected to a light guide rod 50 of the LG connector 18. Therefore, by connecting the light guide rod 50 of the LG connector 18 to the light source device 20, the illumination light emitted from the light source device 20 is transmitted to the illumination windows 22 and 22 via the light guide, and is emitted from the illumination windows 22 and 22. The schematic configuration of the endoscope 10 is as described above.

Next, the configuration of the pipe line of the endoscope 10 will be described.

As shown in FIG. 1, an air/water supply pipe line 52 is inserted into the insertion part 12 of the endoscope 10, and the air/water supply nozzle 46 is connected to an opening of the air/water supply pipe line 52 on the distal end side. The base end side of the air/water supply pipe line 52 is branched into an air supply pipe line 54 and a water supply pipe line 56, and the base end sides of these pipe lines communicate with an inside of a cylinder 58 for air/water supply provided in the hand-side operating part 14. That is, one end side of each of the air supply pipe line 54 and the water supply pipe line 56 communicates with the inside of the cylinder 58, and the other end side of the air supply pipe line 54 and the water supply pipe line 56 is combined to the air/water supply pipe line 52 that is one pipe line.

In addition, the distal end side of each of an air feeding pipe line 60 and a water feeding pipe line 62 communicates with the inside of the cylinder 58, and the air/water supply button 28 is attachably and detachably attached to the inside of the cylinder 58. In a state in which the air/water supply button 28 protrudes, the air supply pipe line 54 and the air feeding pipe line 60 communicate with each other via the cylinder 58, and by performing the pressing operation of the air/water supply button 28, the water supply pipe line 56 and the water feeding pipe line 62 communicate with each other via the cylinder 58. A ventilation hole (not shown) is formed in the air/water supply button 28, and the air feeding pipe line 60 communicates with outside air via the ventilation hole.

The air feeding pipe line 60 and the water feeding pipe line 62 are inserted into the universal cable 16 and are projected toward a water supply connector 64 of the LG connector 18. The pipe line 24 is attachably and detachably connected to the water supply connector 64, and a distal end of the pipe line 24 is connected to a water storage tank 66. The water feeding pipe line 62 communicates below a liquid level of the water storage tank 66, and the air feeding pipe line 60 communicates above the liquid level.

An air pipe line 68 is connected to the water supply connector 64, and the air pipe line 68 communicates with the air feeding pipe line 60. In addition, the air pipe line 68 communicates with an air pump 70 in the light source device 20 by connecting the LG connector 18 to the light source device 20. Therefore, in a case in which the air pump 70 is driven to supply air, the air is supplied into the air feeding pipe line 60 through the air pipe line 68. The air is released to the outside through the ventilation hole (not shown) of the air/water supply button 28 during the non-operation of the air/water supply button 28, but the air in the air feeding pipe line 60 is supplied into the air supply pipe line 54 and the air is jetted from the air/water supply nozzle 46 by blocking the ventilation hole by means of an operator. In addition, in a case in which the pressing operation of the air/water supply button 28 is performed, the air feeding pipe line 60 and the air supply pipe line 54 are blocked, so that the air supplied into the air pipe line 68 is supplied above the liquid level of the water storage tank 66. As a result, the internal pressure of the water storage tank 66 is increased, and water is fed into the water feeding pipe line 62. Then, water is jetted from the air/water supply nozzle 46 through the water supply pipe line 56. In this way, air or water is jetted from the air/water supply nozzle 46, and the observation window 44 is washed by blowing air or water to the observation window 44.

As shown in FIG. 1, a forceps pipe line 72 is inserted into the insertion part 12 of the endoscope 10, a forceps port 48 is opened on the distal end side of the forceps pipe line 72, the base end side of the forceps pipe line 72 branches into two pipe lines 72A and 72B. The base end side of the pipe line 72A communicates with the forceps insertion port 34, and the base end side of the pipe line 72B communicates with the inside of a cylinder 74 for suction. Therefore, in a case in which the treatment tool such as the forceps is inserted from the forceps insertion port 34, the treatment tool can be led out from the forceps port 48.

A base end side of a suction pipe line 76 communicates with the inside of the cylinder 74, and the suction button 30 is attached to the cylinder 74. In a state in which the suction button 30 protrudes, the suction pipe line 76 communicates with the outside air, and the suction pipe line 76 and the forceps pipe line 72 communicate with each other via the pipe line 72B and the cylinder 74 by performing the pressing operation of the suction button 30.

The suction pipe line 76 is projected to a suction connector 78 of the LG connector 18, and the pipe line 26 is connected to the suction connector 78 to communicate with a suction device (not shown). Therefore, by performing the pressing operation of the suction button 30 in a state in which the suction device is driven, a lesion part or the like can be suctioned through the forceps pipe line 72 from the forceps port 48.

As described above, the endoscope 10 comprises a plurality of air/water supply system pipe lines (air feeding pipe line 60, water feeding pipe line 62, cylinder 58, air supply pipe line 54, water supply pipe line 56, and air/water supply pipe line 52) constituting an air/water supply system. The plurality of air/water supply system pipe lines are targets of the washing, and the air/water supply button 28 including the valve member is removable from the cylinder 58 in order to wash the plurality of air/water supply system pipe lines. Similarly, the suction system comprises a plurality of suction system pipe lines (suction pipe line 76, cylinder 74, pipe line 72B, pipe line 72A, and forceps pipe line 72). The plurality of suction system pipe lines are targets of the washing, and the suction button 30 including the valve member is also removable from the cylinder 74 in order to wash the plurality of suction system pipe lines.

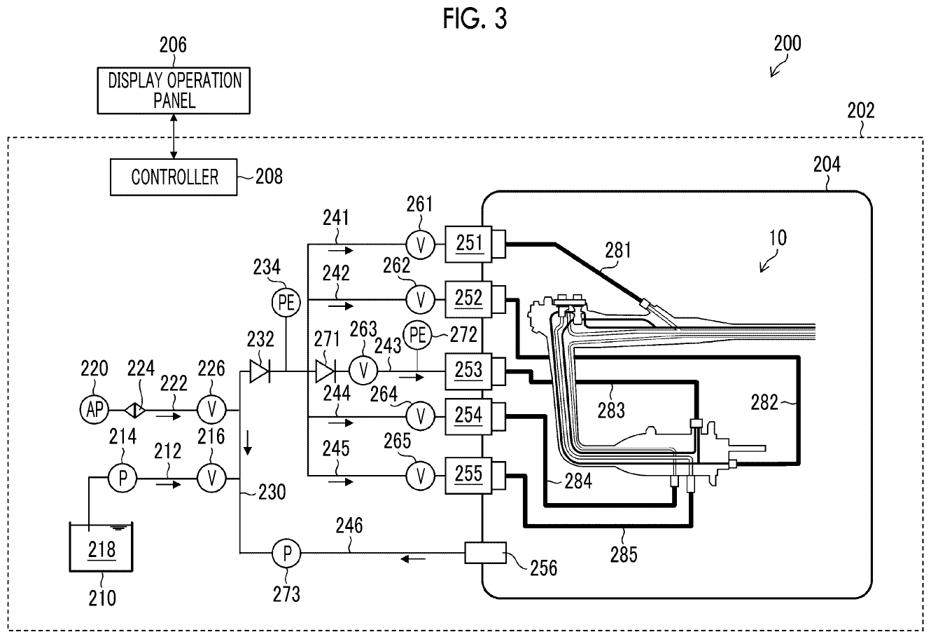

Next, an endoscope washing and disinfection device (hereinafter, referred to as a washing device) 200 according to the embodiment will be described with reference to FIG. 3. FIG. 3 is a block diagram showing a schematic configuration of the washing device 200. FIG. 3 shows a configuration related to the washing of the endoscope pipe line and the determination of the state of the endoscope pipe line, and the detailed configuration of the washing device 200 will be omitted. The washing device 200 can disinfect and wash the air/water supply system pipe line, the suction system pipe line, and other pipe lines (these may be collectively referred to as the endoscope pipe line) of the endoscope 10, and determine the state of the endoscope pipe line.

As shown in FIG. 3, the washing device 200 comprises a box-shaped device body 202, and a washing tank 204 and a display operation panel 206 that are provided on an upper part of the device body 202. The washing tank 204 is a water tank having an open upper part and accommodates the endoscope 10 after use. The washing tank 204 is formed of, for example, a metal having excellent heat resistance, corrosion resistance, and the like, such as stainless steel, and can store a liquid such as a washing solution or a disinfectant solution.

The display operation panel 206 comprises a large number of buttons for various settings related to the washing, the disinfection, and the state determination of the endoscope 10, and giving an instruction to start or stop the washing and the disinfection. The display operation panel 206 comprises, for example, a liquid crystal display, and displays various setting screens, the remaining time of each step, a warning message in a case of trouble, and the like. The display operation panel 206 may be separated into a display panel and an operation panel.

The display operation panel 206 is connected to a controller 208. The controller 208 receives the instruction from the display operation panel 206 and controls the entire washing device 200 according to the instruction. The controller 208 controls the display operation panel 206 to display various types of information.

The washing device 200 comprises a liquid storage tank 210, a liquid supply passage 212 having one end side connected to the liquid storage tank 210, and a pump 214 and a solenoid valve 216 that are disposed in the liquid supply passage 212. The liquid storage tank 210 stores a liquid 218 such as the washing solution, the disinfectant solution, or alcohol. The pump 214 sucks the liquid 218 from the liquid storage tank 210, and supplies the liquid 218 into the liquid supply passage 212. By switching between an opened state and a closed state of the solenoid valve 216, the supply and the stop of the liquid 218 to the liquid supply passage 212 are switched.

The washing device 200 comprises an air pump 220, an air supply passage 222 having one end side connected to the air pump 220, and a filter 224 and a solenoid valve 226 that are disposed in the air supply passage 222. The air pump 220 supplies air into the air supply passage 222 as a gas. The filter 224 is disposed downstream of the air pump 220 and upstream of the solenoid valve 226, and captures miscellaneous bacteria in the air to purify the air. By switching between an opened state and a closed state of the solenoid valve 226, the supply and the stop of the air to the air supply passage 222 are switched.

The washing device 200 comprises a main pipe line 230, and a check valve 232 and a pressure sensor 234 that are disposed in the main pipe line 230. The check valve 232 prevents a reverse flow of the fluid (liquid and gas) in the main pipe line 230. The pressure sensor 234 detects a pressure value of a pressure that is one of physical quantities of the fluid supplied into the main pipe line 230. The pressure sensor 234 is disposed downstream of the check valve 232.

The washing device 200 comprises branch pipe lines 241, 242, 243, 244, and 245, supply ports 251, 252, 253, 254, and 255, and circulation passages 246. The branch pipe lines 241, 242, 243, 244, and 245 are connected to the main pipe line 230 on one end side thereof. The supply ports 251, 252, 253, 254, and 255 are connected to the other end side of each of the branch pipe lines 241, 242, 243, 244, and 245. The supply ports 251, 252, 253, 254, and 255 are disposed in the washing tank 204. The solenoid valves 261, 262, 263, 264, and 265 are respectively disposed in the branch pipe lines 241, 242, 243, 244, and 245. By switching between the opened state and the closed state of the solenoid valves 261, 262, 263, 264, and 265, the supply and the stop of the fluid to each of the branch pipe lines 241, 242, 243, 244, and 245 are switched.

A check valve 271 and a pressure sensor 272 are disposed in the branch pipe line 243. The check valve 271 is disposed upstream of the solenoid valve 263, and the pressure sensor 272 is disposed downstream of the solenoid valve 263. The check valve 271 prevents a reverse flow of the fluid in the branch pipe line 243. The pressure sensor 272 detects a pressure value of a pressure that is one of the physical quantities of the fluid supplied into the branch pipe line 243.

The endoscope 10 shown in FIG. 3 comprises a plurality of pipe lines as in the endoscope 10 shown in FIG. 1. It should be noted that the endoscope 10 shown in FIG. 3 comprises a sub-water supply system pipe line in addition to the suction system pipe line and the air/water supply system pipe line.

Tubes 281, 282, 283, 284, and 285 are respectively connected to the supply ports 251, 252, 253, 254, and 255. The supply port 251 is connected to the suction system pipe line of the endoscope 10 via the tube 281. The supply port 252 is connected to the air/water supply system pipe line of the endoscope 10 via the tube 282. The supply port 253 is connected to the air/water supply system pipe line of the endoscope 10 via the tube 283. The supply port 254 is connected to the sub-water supply system pipe line of the endoscope 10 via the tube 284. The supply port 255 is connected to the suction system pipe line of the endoscope 10 via the tube 285.

A pipe line from a supply source of the fluid to the supply port can be regarded as a supply pipe line in the washing device 200. For example, in a case in which the fluid is a liquid, the liquid supply passage 212, the main pipe line 230, and the branch pipe line 241 that connect the liquid storage tank 210 and the supply port 251 constitute the supply pipe line. In a case in which the fluid is a gas, the air supply passage 222 that connects the air pump 220 and the supply port 251, the main pipe line 230, and the branch pipe line 241 constitute the supply pipe line.

Similarly to the supply port 251, as for the other supply ports 252, 253, 254, and 255, a pipe line from the supply source of the fluid to the supply ports can also be regarded as the supply pipe line in the washing device 200.

As described above, the suction system pipe line, the air/water supply system pipe line, and the sub-water supply system pipe line each constitute the endoscope pipe line of the endoscope 10.

The circulation passage 246 is connected to the main pipe line 230 on one end side thereof. The circulation passage 246 is connected to the main pipe line 230 on a side opposite to a side on which the branch pipe lines 241, 242, 243, 244, and 245 are connected. A circulation port 256 is connected to the other end side of the circulation passage 246. A pump 273 is disposed in the circulation passage 246. The pump 273 sucks the liquid of the washing tank 204 from the circulation port 256, and supplies the liquid into the main pipe line 230.

The solenoid valves 216, 226, 261, 262, 263, 264, and 265 are connected to the controller 208, and the controller 208 switches between the opened state and the closed state of each of the solenoid valves 216, 226, 261, 262, 263, 264, and 265.

The pumps 214 and 273 and the air pump 220 are connected to the controller 208, and the controller 208 controls the driving of the pumps 214 and 273 and the air pump 220.

The pressure sensors 234 and 272 are connected to the controller 208, and the controller 208 is configured to acquire the pressure values of the fluid detected by the pressure sensors 234 and 272. The pressure sensors 234 and 272 are examples of a physical quantity detection sensor.

It should be noted that the controller 208 comprises an operation circuit formed by various processors, a memory, and the like. The various processors include a central processing unit (CPU), a graphics processing unit (GPU), an application specific integrated circuit (ASIC), and a programmable logic device [for example, simple programmable logic devices (SPLD), complex programmable logic device (CPLD), and field programmable gate arrays (FPGA)]. It should be noted that various functions of the controller 208 may be realized by one processor or a plurality of processors of the same type or different types.

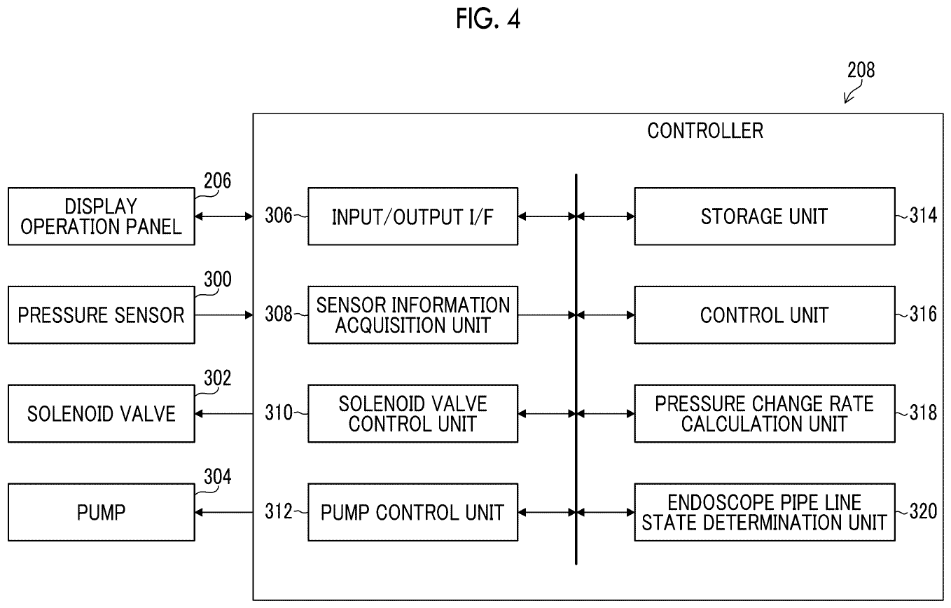

Next, a schematic configuration of the controller 208 will be described. FIG. 4 is a block diagram showing the schematic configuration of the controller (referred to as a control device) 208 of the washing device 200. The display operation panel 206, a pressure sensor 300, a solenoid valve 302, and a pump 304 are connected to the control device 208. The pressure sensor 300 corresponds to, for example, the pressure sensors 234 and 272 (see FIG. 3) disposed in the washing device 200. The solenoid valve 302 corresponds to the solenoid valves 216, 226, 261, 262, 263, 264, and 265 disposed in the washing device 200. The pump 304 corresponds to the pumps 214 and 273 and the air pump 220 disposed in the washing device 200.

The control device 208 mainly comprises an input/output interface (I/F) 306, a sensor information acquisition unit 308, a solenoid valve control unit 310, a pump control unit 312, a storage unit 314, a control unit 316, a pressure change rate calculation unit 318, and an endoscope pipe line state determination unit 320, and executes a control program (not shown) read out from the storage unit 314 to realize each function and execute processing. The control unit 316 controls the overall processing of the control device 208.

The input/output interface 306 can input various data (information) to the washing device 200 via the display operation panel 206, and can output various data (information) from the washing device 200. The input/output interface 306 can perform input/output of data with a network other than the display operation panel 206, other devices, and the like via wired and wireless communication.

The sensor information acquisition unit 308 acquires the pressure value detected by the pressure sensor 300. The sensor information acquisition unit 308 is configured to acquire a physical quantity other than the pressure value detected by the pressure sensor 300, for example, a flow rate value in a case in which a flow rate sensor is provided. That is, the sensor information acquisition unit 308 is configured depending on the physical quantity to be acquired.

The solenoid valve control unit 310 switches between the opened state and the closed state of the solenoid valve 302 based on a control signal from the control unit 316. The pump control unit 312 controls the rotation speed of the pump 304 and the like based on the control signal from the control unit 316, to control the supply amount of the fluid.

The storage unit 314 stores the control program used for controlling the entire washing device 200, a control program used for determining the state of the endoscope pipe line, various types of control information, a past usage status, and the like.

The pressure change rate calculation unit 318 calculates a change rate of the pressure based on the pressure value detected by the pressure sensor 300 and acquired by the sensor information acquisition unit 308, as will be described below.

The endoscope pipe line state determination unit 320 determines the state of the endoscope pipe line based on the change rate calculated by the pressure change rate calculation unit 318, as will be described below.

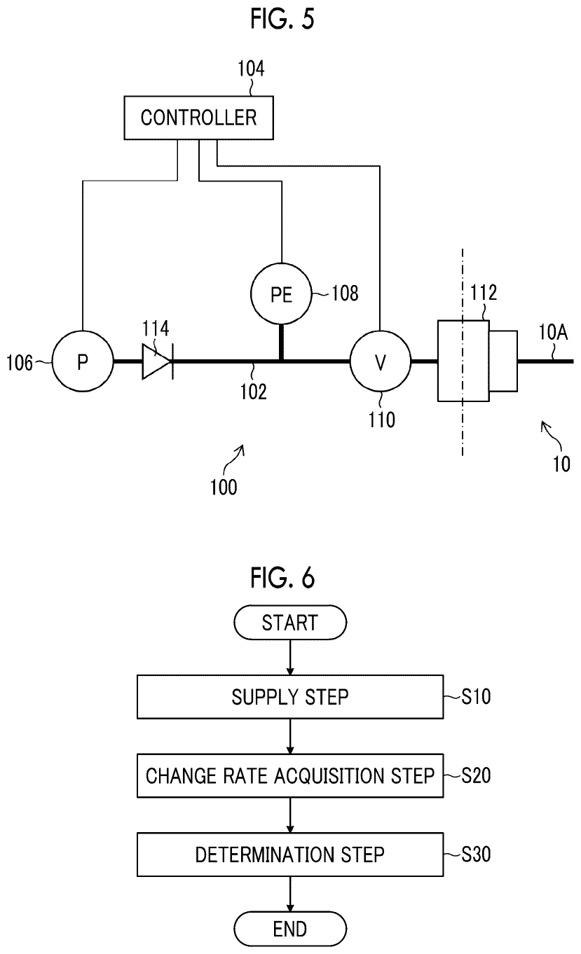

Hereinafter, a state determination device 100 according to the embodiment will be described. It should be noted that the state determination device 100 is included in the washing device 200 (realized by the constituent elements of the washing device 200), and hereinafter, a device comprising the constituent elements necessary for the state determination of the endoscope pipe line will be referred to as the state determination device. FIG. 5 shows a connection configuration between the state determination device 100 and an endoscope pipe line 10A of the endoscope 10. The state determination device 100 comprises a supply pipe line 102 through which the fluid is supplied, a controller 104, a pump 106 that supplies the fluid, a pressure sensor 108 that detects the pressure of the fluid, a solenoid valve 110 that switches between the opened state and the closed state to switch between the supply and the stop of the fluid, a supply port 112, and a check valve 114. The supply pipe line 102 of the state determination device 100 and the endoscope pipe line 10A of the endoscope 10 are connected to each other via the supply port 112. The supply pipe line 102 and the endoscope pipe line 10A may be connected to each other via a tube in addition to the supply port 112.

The supply pipe line 102 corresponds to the supply pipe line described with reference to FIG. 3, the controller 104 corresponds to the controller 208 described with reference to FIG. 4, the pump 106 corresponds to the pump 304 described with reference to FIG. 4, the pressure sensor 108 corresponds to the pressure sensor 300 described with reference to FIG. 4, the solenoid valve 110 corresponds to the solenoid valve 302 described with reference to FIG. 4, the check valve 114 corresponds to the check valves 232 and 271 described with reference to FIG. 3, and the supply port 112 corresponds to the supply ports 251 to 255 described with reference to FIG. 3. The endoscope pipe line 10A corresponds to the suction system pipe line, the air/water supply system pipe line, and the sub-water supply system pipe line described with reference to FIG. 3.

Next, the state determination method for the endoscope pipe line will be described. The endoscope 10 is washed, for example, by connecting the supply pipe line 102 and the endoscope pipe line 10A via the supply port 112, driving the pump 106 with the solenoid valve 110 in the opened state, supplying the fluid into the supply pipe line 102, and supplying the fluid from the supply port 112 into the endoscope pipe line 10A. The washing of the endoscope 10 is carried out, for example, by a washing step, a disinfection step, and a rinsing step, and the inside of the endoscope pipe line 10A is washed by flowing a predetermined amount of the fluid such as the washing solution, the disinfectant solution, or water in the endoscope pipe line 10A. However, in a case in which the predetermined amount of the fluid does not flow through the endoscope pipe line 10A, that is, in a case in which the endoscope pipe line 10A is blocked, sufficient washing cannot be performed, and the fluid cannot be supplied or sucked during use. Therefore, it is important to perform the state determination of the endoscope pipe line 10A before performing the washing of the endoscope 10.

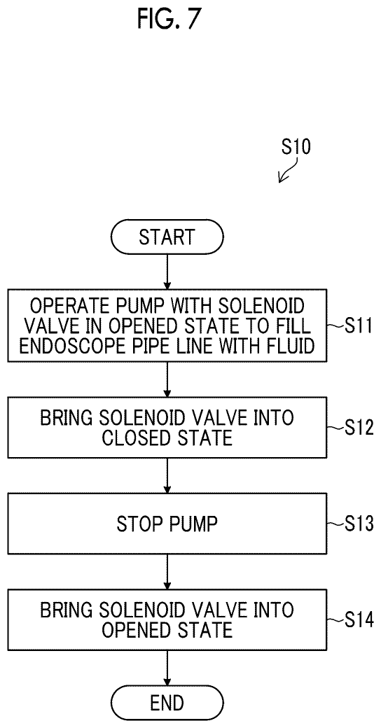

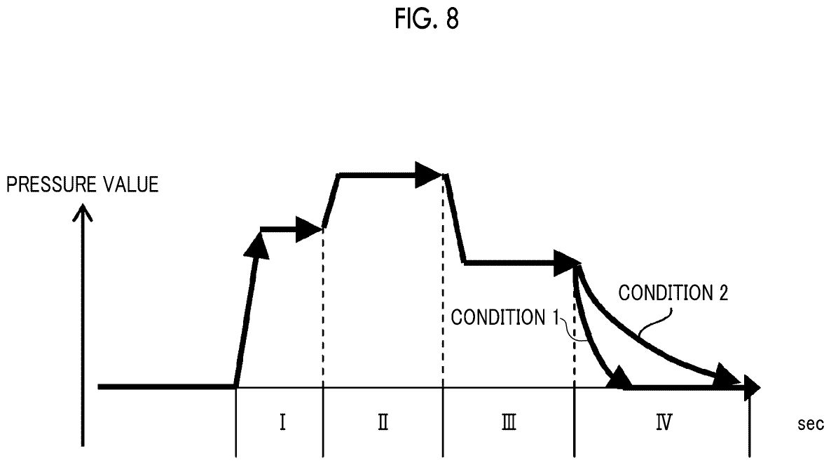

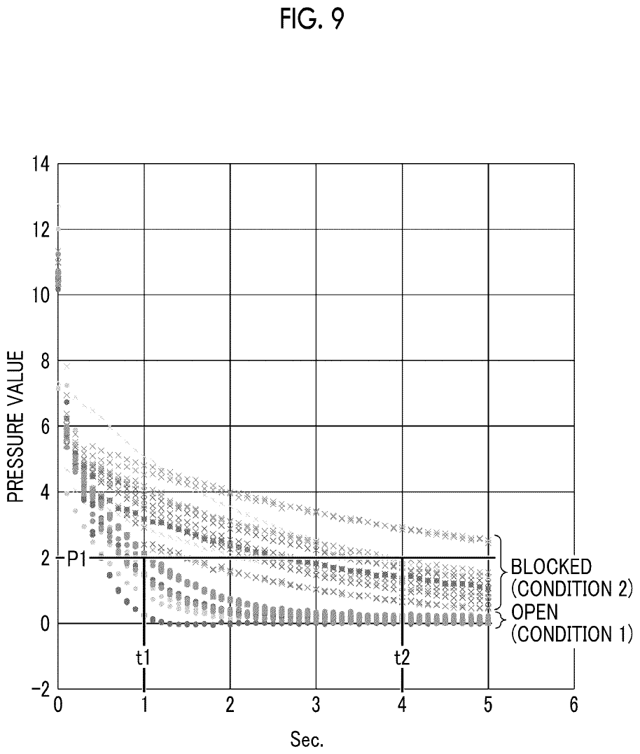

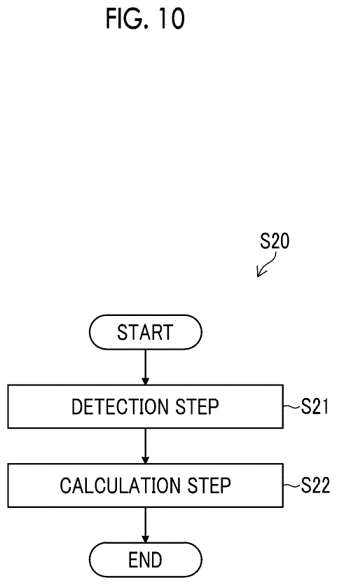

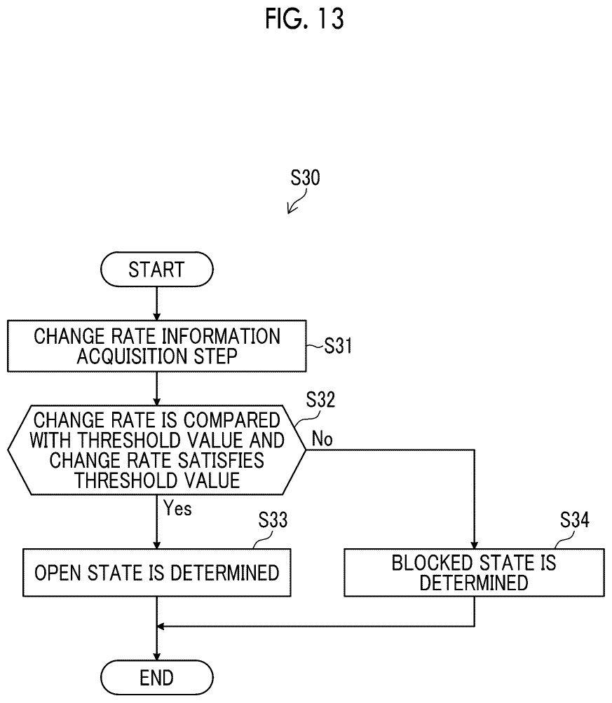

FIG. 6 is a flowchart of the state determination method for the endoscope pipe line. FIG. 7 is a flowchart of a supply step. FIG. 8 is a graph showing a change in the pressure in the supply pipe line in the supply step shown in FIG. 7. FIG. 9 is a graph of an actual pressure value for a determination period of FIG. 8. FIG. 10 is a flowchart of a change rate acquisition step. FIG. 13 is a flowchart of a determination step.

As shown in FIG. 6, the state determination method for the endoscope pipe line includes the supply step (step S10), the change rate acquisition step (step S20), and the determination step (step S30). The supply step (step S10) is a step of supplying the pressurized fluid into the endoscope pipe line 10A. The change rate acquisition step (step S20) is a step of acquiring a change rate, which is a change amount per unit time of the pressure of the fluid in the determination period after the supply of the fluid is stopped in the supply step. The determination step (step S30) is a step of determining the state (open state and blocked state) of the endoscope pipe line 10A based on the change rate acquired in the change rate acquisition step. Hereinafter, each step will be described.

### <Supply Step (Step S10)>

As shown in FIG. 7, the supply step (step S10) comprises, as an example, a step (step S11) of operating the pump with the solenoid valve in the opened state, to fill the endoscope pipe line with the fluid, a step (step S12) of bringing the solenoid valve into the closed state, a step (step S13) of stopping the pump, and a step (step S14) of bringing the solenoid valve into the opened state.

In the supply step (step S10), in the state determination device 100 shown in FIG. 5, the solenoid valve 110 is brought into the opened state, the pump 106 is operated, and the fluid is supplied from the supply pipe line 102 into the endoscope pipe line 10A to fill the endoscope pipe line 10A with the fluid (step S11).

In step S11, the solenoid valve 110 is brought into the opened state to operate the pump 106 based on the control signal from the controller 104. The fluid is supplied into the supply pipe line 102 by the pump 106. The supply pipe line 102 supplies the fluid into the endoscope pipe line 10A via the supply port 112. By operating the pump 106 with the solenoid valve 110 in the opened state, the supply pipe line 102 and the endoscope pipe line 10A are filled with the fluid with the lapse of time.

FIG. 8 shows a change in the pressure in step S11 in a period I. As shown in FIG. 8, in a case in which the pump 106 is operated and the fluid is supplied into the supply pipe line 102 and the endoscope pipe line 10A, the pressure of the fluid in the supply pipe line 102 is increased. The pressure of the fluid in the supply pipe line 102 is detected by the pressure sensor 108. The pressure is increased until the insides of the supply pipe line 102 and the endoscope pipe line 10A are filled with the fluid. The endoscope pipe line 10A has a certain pressure after the inside of the endoscope pipe line 10A is filled with the fluid.

Next, as shown in FIG. 7, the solenoid valve 110 is brought into the closed state (step S12). In step S12, the solenoid valve 110 is in the closed state based on the control signal from the controller 104. The solenoid valve 110 is in the closed state, while the pump 106 continues to operate to supply the fluid into the supply pipe line 102.

FIG. 8 shows a change in the pressure in step S12 in a period II. Since the solenoid valve 110 is in the closed state and the pump 106 supplies the fluid into the supply pipe line 102, as shown in FIG. 8, the pressure in the supply pipe line 102 is increased as compared with the pressure in step S11, and reaches a certain pressure.

Next, as shown in FIG. 7, the pump 106 is stopped (step S13). After the pressure in step S12 is converted into a constant, in step S13, the pump 106 is stopped based on the control signal from the controller 104. The solenoid valve 110 is in the closed state, and the pump 106 is stopped.

FIG. 8 shows a change in the pressure in step S13 in a period III. The pump 106 is stopped, and the supply of the fluid is stopped, so that the pressure in the supply pipe line 102 decreases as shown in FIG. 8. Meanwhile, in the supply pipe line 102, a state in which the fluid is pressurized at a certain pressure is maintained, that is, a so-called pressurized state is obtained. The fluid is stored in the supply pipe line 102.

Next, as shown in FIG. 7, the solenoid valve 110 is brought into the opened state (step S14). In step S14, the solenoid valve 110 is brought into the opened state based on the control signal from the controller 104. As a result, the pressurized fluid from the supply pipe line 102 is supplied into the endoscope pipe line 10A via the supply port 112. In a case in which the pump 106 is in the stop state and the pressurized fluid is supplied into the endoscope pipe line 10A, the fluid pressure of the fluid in the supply pipe line 102 changes.

FIG. 8 shows a change in the pressure in step S14 in a period IV. Since the pump 106 is in the stop state and the solenoid valve 110 is in the opened state, the pressure in the supply pipe line 102 decreases as shown in FIG. 8. The controller 104 determines the state of the endoscope pipe line 10A based on the change (attenuation) in the pressure during the period in which the pressurized fluid is supplied into the endoscope pipe line 10A. Here, the period IV in FIG. 8 corresponds to the determination period, and the continuous supply of the fluid by the pump 106 is stopped in the determination period. The change rate of the physical quantity in the supply pipe line 102 in the determination period is acquired, and the state of the endoscope pipe line 10A is determined based on the change rate.

For example, in a case in which the endoscope pipe line 10A is in the open state, the pressure in the supply pipe line 102 rapidly decreases as shown in a condition 1. On the other hand, in a case in which the endoscope pipe line 10A is in the blocked state, the pressure does not decrease in a case in which there is no other portion that leaks. However, in a case in which the fluid is intentionally leaked at the connection portion between the endoscope pipe line 10A and the tube, the pressure gradually decreases as in a condition 2 even in a case in which the endoscope pipe line 10A is in the blocked state. The change (transition) in the attenuation of the pressure value is generally different between the open state (condition 1) and the blocked state (condition 2) of the endoscope pipe line 10A, so that it is considered that the open state and the blocked state of the endoscope pipe line 10A can be determined from the change (transition) in the attenuation of the pressure value.

FIG. 9 is an actual graph of the pressure value in a time in the period IV (determination period) of FIG. 8. A horizontal axis indicates a time, and a vertical axis indicates a pressure value. The graph shows the pressure values detected by the pressure sensor 108 corresponding to a plurality of time points within the determination period.

The graph shows a change (transition) in the pressure value including the open state (condition 1) and the blocked state (condition 2). According to the change (transition) in an ideal pressure value shown in FIG. 8, it is also considered that the open state (condition 1) and the blocked state (condition 2) can be determined.

However, in a change (transition) in the pressure value shown in FIG. 9, the determination is substantially difficult. In a case in which the length or the thickness of the endoscope pipe line 10A, or the pressure before the opened state of the solenoid valve 110 in step S13 varies, the change (transition) in the attenuation is affected. The graph of FIG. 9 plots the changes (transitions) in the pressure values for dozens of data series in which these conditions are different. In reality, half of dozens of data series belong to the open state (condition 1), and the remaining half of the data series belong to the blocked state (condition 2). For example, in a case in which the times at which the pressure reaches P1 are compared, even in the same blocked state (condition 2), there is a variation like t1 and t2. At t1, the open state (condition 1) is almost the same. Therefore, it is difficult to determine the open state or the blocked state of the endoscope pipe line 10A from the change (transition) in the pressure value with time.

In view of the above, the present inventors have intensively studied the problem, and have found that it is effective to determine the state (open state or blocked state) of the endoscope pipe line 10A based on a change rate by focusing on a change rate, which is a change amount of the physical quantity per unit time, instead of a change (transition) in the physical quantity such as the pressure value, and this has led to the present invention.

Hereinafter, the change rate acquisition step (step S20) and the determination step (step S30) will be described.

### <Change Rate Acquisition Step (Step S20)>

Next, the change rate acquisition step (step S20) will be described with reference to the flowchart of the change rate acquisition step of FIG. 10. The change rate acquisition step (step S20) comprises, as an example, a detection step (step S21) and a calculation step (step S22).

In the detection step, physical quantity data indicating a physical quantity of the fluid corresponding to each of the plurality of time points within the determination period is detected (step S21). In step S21, the state determination device 100 detects the pressure value indicating the pressure that is the physical quantity of the fluid corresponding to each of the plurality of time points within the determination period by the pressure sensor 108 as the physical quantity data. The pressure value detected by the pressure sensor 108 is acquired by the sensor information acquisition unit 308.

The sensor information acquisition unit 308 acquires the pressure value as the pressure in the supply pipe line 102, for example, as shown in the graph of FIG. 9.

In the calculation step, the change rate (pressure change rate), which is the change amount per unit time of the pressure value, is calculated based on the pressure value, which is the physical quantity data detected in the detection step (step S22). In step S22, the pressure change rate calculation unit 318 calculates the pressure change rate based on the pressure value acquired by the sensor information acquisition unit 308 (that is, the pressure value detected by the pressure sensor 108 in step S21). It should be noted that, in the embodiment, since the pressure value detected by the pressure sensor 108 gradually decreases with the lapse of time, the pressure change rate can also be referred to as a decrease rate per unit time of the pressure value.

The pressure change rate calculation unit 318 performs conversion processing of converting the change rate into a constant such that the change rate can be compared with a predetermined threshold value (for example, a threshold value that is a constant). Converting the change rate into a constant means that the change rate approaches a predetermined constant, for example, a slope of the change rate approaches a straight line. The conversion processing is not limited as long as the change rate can be converted into a constant, and includes any processing. The conversion processing includes, for example, appropriately setting a range such that the change rate is approximately constant, performing conversion processing of converting the change rate into a constant in order to compare the change rate with one threshold value, and performing time division and arithmetic processing of excluding a range that is unstable or eliminating an outlier or a variation in order to eliminate erroneous detection during the conversion processing.

Next, an example of the conversion processing of converting the change rate into a constant will be described. As an example of the conversion processing, there is an example in which the change rate is converted into a constant by performing a logarithmic conversion on at least one of the physical quantity data or time data indicating the elapsed time from the start of the determination period.

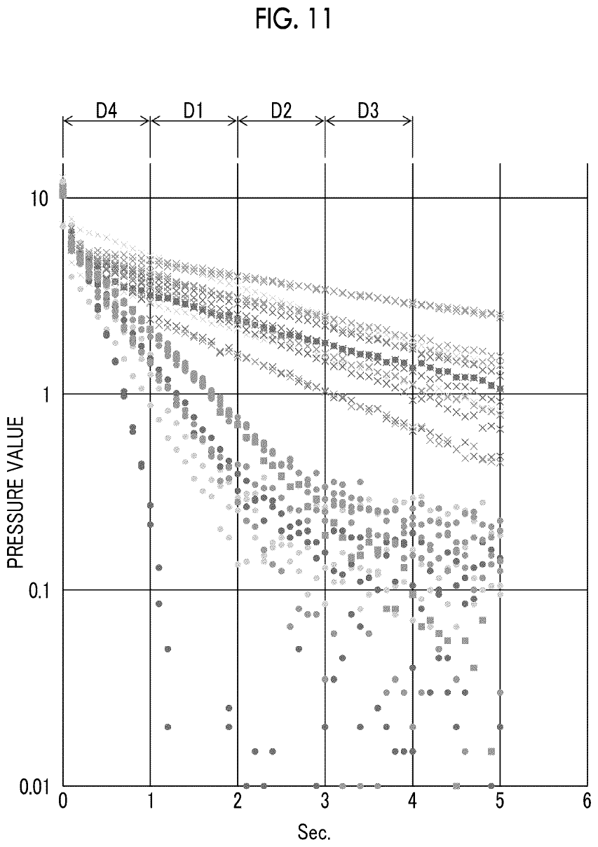

FIG. 11 is a semi-logarithmic graph in which a vertical axis is a logarithmic axis of the pressure value and a horizontal axis is a time axis. FIG. 11 is a graph with the pressure value on the logarithmic axis for the change (transition) of pressure shown in FIG. 9 in which the horizontal axis indicates the time data indicating the elapsed time from the start of the determination period, and the vertical axis indicates the pressure value which is the physical quantity data, and the conversion processing is performed such that the change rate of the physical quantity data is substantially constant.

In a case in which the pressure value and the time have a relationship as shown in the graph of FIG. 11, even in a case in which (1) the waveform during the attenuation of the pressure is disturbed due to the influence of the variation in a measurement device, the turbulence of the pipe line, or the like, or (2) the outlier is generated due to the influence of the noise or the like, the relationship between the pressure value and the time is easily read.

It should be noted that, in FIG. 11, the vertical axis showing the pressure value of the physical quantity data in FIG. 9 is logarithmically converted, but the time axis may be logarithmically converted or both axes may be logarithmically converted as long as the change rate can be converted into a constant.

Although the logarithmic conversion is shown as the conversion processing in FIG. 11, a time interval (horizontal axis) may be appropriately changed, a physical quantity interval (vertical axis) may be appropriately changed, or these may be combined and appropriately changed as long as the change rate can be converted into a constant. That is, the unit interval of each parameter (time and physical quantity) may be an equal interval or a logarithmic interval, may be any predetermined interval, and may be selected according to the characteristics of the change rate.

The state determination device 100 may prepare a constant pattern of a combination of several time intervals (horizontal axis) at which the change rate can be converted into a constant and the physical quantity interval (vertical axis), and may apply the physical quantity data to the constant pattern to convert the change rate into the constant in a case in which the physical quantity data during the determination period of the supply pipe line 102 is detected.

Although the pressure value is shown as the physical quantity data in FIGS. 9 and 11, a flow rate of the supply pipe line 102 may be used as the physical quantity data. In this case, the state determination device 100 of FIG. 5 is provided with a flow rate sensor that detects the flow rate of the fluid flowing through the supply pipe line 102, and the sensor information acquisition unit 308 acquires the detection value of the flow rate sensor.

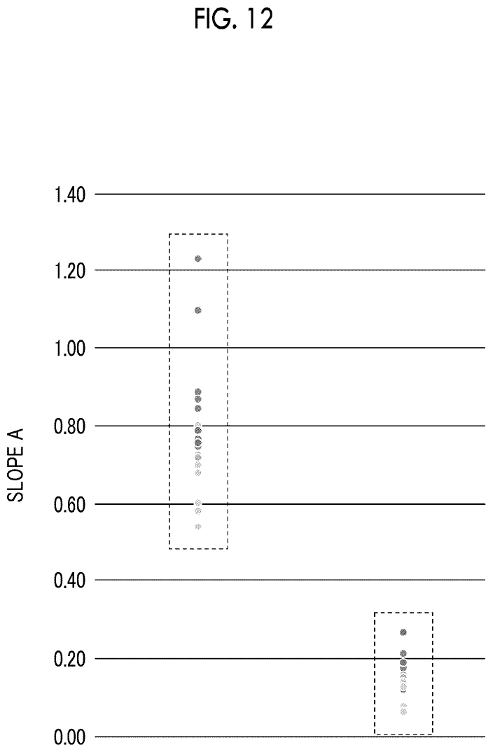

In the calculation step, in order to facilitate comparison with a predetermined threshold value (constant), processing of converting the change rate that is approximately constant into a constant may be included. FIG. 12 is a graph obtained by performing the conversion processing on the change rate of the plurality of data series for which the change rate is substantially constant as shown in the graph of FIG. 11, such that a slope A of each data series is converted into a constant. As a result of the conversion processing, the change rate can be expressed as the slope A (constant). As shown in FIG. 12, it can be understood that the slope A is divided into two point-sets according to the magnitude thereof. It can be said that the two point-sets are formed such that the state (open state and blocked state) of the endoscope pipe line 10A is reflected in the magnitude of the change rate (slope A) of the pressure value of the supply pipe line 102. On the contrary, the state (open state and blocked state) of the endoscope pipe line 10A can be determined according to the magnitude of the change rate (slope A) of the pressure value of the supply pipe line 102, in a case in which the two point-sets can be formed.

As an example of the calculation step, FIG. 12 shows a case in which the change rate that is substantially constant as shown in the graph of FIG. 11 is converted into a constant by performing the conversion processing. Hereinafter, preferred aspects will be described. In the calculation step, the following preferred aspects are applied alone or in combination by the pressure change rate calculation unit 318.

In the calculation step, for example, the change rate can be calculated based on time-division data obtained by time-dividing the physical quantity data for each time, for the graph of FIG. 11. In FIG. 11, the physical quantity data need not be continuous in time, and the change rate that is converted into the constant as shown in FIG. 12 can be acquired as long as the physical quantity data is acquired at time intervals such that the change rate can be acquired.

In the calculation step, for example, (1) the change rate can be calculated by performing linear approximation of the time-division data, on the graph of FIG. 11. In a case in which the linear approximation is possible, the slope A of the change rate can be acquired as a constant.

In the calculation step, for example, (2) the change rate can be calculated based on the slope between two points included in the time-division data for the graph of FIG. 11. Since the slope is a slope between two points, the slope A of the change rate can be acquired as a constant.

In the calculation step, for example, (3) the change rate can be calculated by performing linear approximation based on the residual of the time-division data, on the graph of FIG. 11. In a case in which the linear approximation is possible, the slope A of the change rate can be acquired as a constant.

In the calculation step, for example, (4) the change rate can be calculated by performing linear approximation in which a sum of squares of residuals of the time-division data is minimized, on the graph of FIG. 11. The graph shown in FIG. 12 shows a result of performing the linear approximation in which a sum of squares of residuals of the time-division data is minimized, on the graph of FIG. 11. It can be understood that the slope A of the change rate can be acquired as a constant.

It is preferable that the calculation step comprises, for example, (5) an outlier exclusion step of specifying an outlier included in the physical quantity data based on the physical quantity data after the conversion processing is performed, to exclude the outlier from the physical quantity data.

For example, in a case in which the graph shown in FIG. 9 in which the vertical axis is the pressure value and the horizontal axis is the time is subjected to the conversion processing into the graph in which the change rate shown in FIG. 11 is substantially constant, it is preferable to exclude the maximum value and the minimum value as the outliers from the pressure values in the same data series. In a case of performing the conversion processing from the semi-logarithmic graph in FIG. 11 in which the vertical axis is the pressure value and the horizontal axis is the time into the graph of the slope A of the change rate shown in FIG. 12 by excluding the outlier from the data series, the correct slope A of the change rate can be obtained, and as a result, erroneous detection can be suppressed.

It is preferable that the calculation step comprises (6) a variation determination step of determining a degree of a variation in the physical quantity data based on the physical quantity data after the conversion processing is performed. In the variation determination step, for example, a threshold value is set in advance, and it can be determined whether or not there is an error in the physical quantity data based on the threshold value. In a case in which there is the error, it is possible to specify a cause and suppress erroneous detection such as re-measurement at an early stage.

In the calculation step, as shown in FIG. 11, the determination period may be divided into D1, D2, and D3, the conversion processing of (1) to (6) in the calculation step may be performed in the ranges of the divided periods D1, D2, and D3, and the conversion processing may be performed such that the slope A of the change rate of the data series takes a constant value as shown in FIG. 12.

It is preferable that, in the calculation step, the determination period is a period after a predetermined exclusion period has elapsed from the stop of the supply of the fluid.

As shown in FIG. 11, a period after the exclusion period D4 has elapsed may be used as the determination period and subjected to the conversion processing of (1) to (6) in the calculation step, and the conversion processing may be performed such that the slope A of each data series takes a constant value as shown in FIG. 12. For example, the exclusion period D4 is a certain period after the solenoid valve in step S14 is brought into the opened state. In this certain period, a change point exists in the change (transition) in the pressure value, and a position of the change point varies depending on the model of the endoscope or the open state of the pipe line. By not including the pressure value for this certain period, a range in which the physical quantity data is likely to be unstable can be excluded. Therefore, in a case in which the conversion processing is performed such that the slope A of the pressure change rate of each data series takes a constant value, the correct slope A can be obtained, and as a result, erroneous detection can be suppressed.

### <Determination Step (Step S30)>

Next, the determination step (step S30) will be described with reference to FIG. 13. The determination step (step S30) is a step of determining the state of the endoscope pipe line, and determining whether the endoscope pipe line is in the open state or the blocked state as the state of the endoscope pipe line.

The determination step (step S30) comprises a change rate information acquisition step (step S31) of acquiring the calculated change rate, a comparison step (step S32) of comparing the change rate with the threshold value, an open state determination step (step S33) of determining that the endoscope pipe line 10A is in the open state, and a blocked state determination step (step S34) of determining that the endoscope pipe line 10A is in the blocked state. It should be noted that the threshold value used in the comparison step is an example of a determination threshold value indicating whether the endoscope pipe line is open or blocked.

In the change rate information acquisition step, the change rate calculated in the change rate acquisition step (step S20) is acquired (step S31). In step S31, the endoscope pipe line state determination unit 320 of the state determination device 100 acquires the change rate (slope A) calculated by the pressure change rate calculation unit 318.

In the comparison step of comparing the change rate with the threshold value, the change rate (slope A) acquired in step S31 is compared with the threshold value set in advance, and it is determined whether or not the change rate (slope A) satisfies the threshold value (step S32). In step S32, the endoscope pipe line state determination unit 320 compares the acquired change rate (slope A) with the threshold value, and determines whether or not the threshold value is satisfied. In a case in which the endoscope pipe line state determination unit 320 determines that the threshold value is satisfied, the process proceeds to step S33, and the endoscope pipe line 10A is determined to be in the open state. On the other hand, in a case in which the endoscope pipe line state determination unit 320 determines that the threshold value is not satisfied, the process proceeds to step S34, and it is determined that the endoscope pipe line 10A is in the blocked state. The result information of the determination step is transmitted to, for example, the control unit 316. The control unit 316 stores the result information in the storage unit 314, and displays the result information on the display operation panel 206 via the input/output I/F. The determination step ends in this manner.

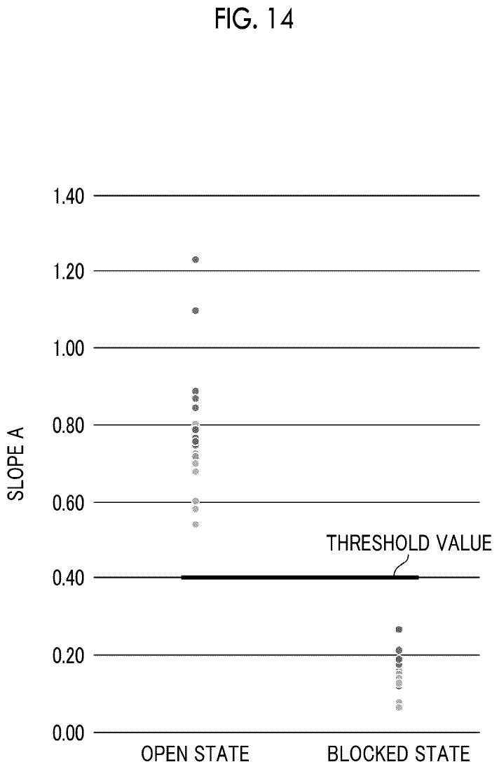

FIG. 14 is a diagram showing the comparison between the change rate and the threshold value, and is a diagram in which the threshold value and the state of the endoscope pipe line are added to FIG. 12. It should be noted that the vertical axis in FIG. 14 shows the absolute value of the change rate (slope A).

The endoscope pipe line state determination unit 320 determines the state of the endoscope pipe line (open state and blocked state) by comparing the predetermined threshold value with the change rate (slope A). For example, in the example of FIG. 14, in a case in which the change rate (slope A) is larger than the threshold value, the endoscope pipe line state determination unit 320 determines that the threshold value is satisfied and that the state of the endoscope pipe line 10A is the open state. On the other hand, in a case in which the change rate (the slope A) is smaller than the threshold value, the endoscope pipe line state determination unit 320 determines that the threshold value is not satisfied and that the state of the endoscope pipe line 10A is the blocked state.

As shown in FIG. 14, since the time-divided physical quantity data is defined as a constant such as the slope A, and this constant is compared with a predetermined threshold value (constant), it is easy to detect an abnormal point such as a waveform disturbance or an outlier. The influence of the variation in the initial value and the like can be excluded. The endoscope pipe line state can be determined with high accuracy.

In FIG. 14, the case has been described in which one threshold value is set in advance, but a blocking determination threshold value for determining the blocked state and an opening determination threshold value for determining the open state may be separately set. The threshold value in FIG. 14 serves as both the blocking determination threshold value and the opening determination threshold value.

In FIG. 14, as an example, the case has been described in which the absolute value of the change rate (slope A) is taken and compared with the threshold value, but the present invention is not limited to this, and a case may be adopted in which the absolute value of the change rate (slope A) is not taken and the comparison with the threshold value is performed. In any case, by appropriately determining the threshold value for determining whether the endoscope pipe line is in the open or blocked state, it is possible to determine whether the endoscope pipe line is in the open or blocked state.

It should be noted that, in the embodiment described above, as one of preferred aspects, the aspect in which the physical quantity of the fluid according to the embodiment of the present invention is the pressure or the flow rate of the fluid has been described, but the present invention is not limited to this, and for example, an aspect (first modification example) in which the physical quantity of the fluid is a temperature of the fluid and an aspect (second modification example) in which the physical quantity of the fluid is a flow velocity (kinetic energy) of the fluid can be adopted.

In the first modification example, a flow temperature sensor (not shown) provided in the supply pipe line 102 detects a temporal change in the temperature of the fluid flowing through the supply pipe line 102, and whether the endoscope pipe line is in the open or blocked state is determined based on a temperature change rate, which is a change amount per unit time of the temperature. In this case, in the temporal change in the temperature of the fluid detected by the temperature sensor, the temperature of the fluid preferably gradually decreases with the lapse of time, and the temperature change rate is the decrease rate of the temperature per unit time. In a case in which the temperature of the fluid gradually increases with the lapse of time, the temperature change rate is an increase rate per unit time of the temperature.

In the second modification example, a flow velocity sensor (not shown) provided in the supply pipe line 102 detects a temporal change in the velocity of the fluid flowing through the supply pipe line 102, and whether the endoscope pipe line is in the open or blocked state is determined based on a velocity change rate, which is a change amount per unit time of the velocity. In this case, in the temporal change in the velocity of the fluid detected by the flow velocity sensor, the velocity of the fluid preferably gradually increases with the lapse of time, and the velocity change rate is the increase rate of the flow velocity per unit time. In a case in which the velocity of the fluid gradually decreases with the lapse of time, the velocity change rate is a decrease rate per unit time of the flow velocity.

### EXPLANATION OF REFERENCES

10: endoscope
10A: endoscope pipe line
12: insertion part
14: hand-side operating part
16: universal cable
18: LG connector
20: light source device
22: illumination window
24: pipe line
26: pipe line
28: air/water supply button
30: suction button
32: shutter button
34: forceps insertion port
36: distal end part
38: bendable part
40: soft part
42: distal end surface
44: observation window
46: air/water supply nozzle
48: forceps port
50: light guide rod
52: air/water supply pipe line
54: air supply pipe line
56: water supply pipe line
58: cylinder
60: air feeding pipe line
62: water feeding pipe line
64: water supply connector
66: water storage tank
68: air pipe line
70: air pump
72: forceps pipe line
72A: pipe line
72B: pipe line
74: cylinder
76: suction pipe line
78: suction connector
100: state determination device
102: supply pipe line
104: controller
106: pump
108: pressure sensor
110: solenoid valve
112: supply port
114: check valve
200: endoscope washing and disinfection device
202: device body
204: washing tank
206: display operation panel
208: control device
208: controller
210: liquid storage tank
212: liquid supply passage
214: pump
216: solenoid valve
218: liquid
220: air pump
222: air supply passage
224: filter
226: solenoid valve
230: main pipe line
232: check valve
234: pressure sensor
241: branch pipe line
242: branch pipe line
243: branch pipe line
244: branch pipe line
245: branch pipe line
246: circulation passage
251: supply port
252: supply port
253: supply port
254: supply port
255: supply port
256: circulation port
261: solenoid valve
262: solenoid valve
263: solenoid valve
264: solenoid valve
265: solenoid valve
271: check valve
272: pressure sensor
273: pump
281: tube
282: tube
283: tube
284: tube
285: tube
300: pressure sensor
302: solenoid valve
304: pump
306: input/output interface
308: sensor information acquisition unit
310: solenoid valve control unit
312: pump control unit
314: storage unit
316: control unit
318: pressure change rate calculation unit
320: endoscope pipe line state determination unit

What is claimed is:

1. A state determination method for an endoscope pipe line, comprising:
a supply step of supplying a pressurized fluid into an endoscope pipe line;
a change rate acquisition step of acquiring a change rate, which is a change amount per unit time of a physical quantity of the fluid within a determination period after the supply of the fluid is stopped; and
a determination step of determining whether the endoscope pipe line is in an open or blocked state based on the change rate acquired in the change rate acquisition step.

2. The state determination method for an endoscope pipe line according to claim 1,
wherein the physical quantity is a pressure or a flow rate of the fluid.

3. The state determination method for an endoscope pipe line according to claim 1,
wherein the supply of the fluid is stopped after the endoscope pipe line is filled with the fluid in the supply step.

4. The state determination method for an endoscope pipe line according to claim 1,
wherein the change rate acquisition step includes
a detection step of detecting physical quantity data indicating a physical quantity of the fluid corresponding to each of a plurality of time points within the determination period, and
a calculation step of calculating the change rate based on the physical quantity data detected in the detection step.

5. The state determination method for an endoscope pipe line according to claim 4,
wherein the calculation step includes conversion processing of converting the change rate into a constant.

6. The state determination method for an endoscope pipe line according to claim 5,
wherein, in the calculation step, as the conversion processing, logarithmic conversion is performed on at least one of the physical quantity data or time data indicating an elapsed time from a start of the determination period, to convert the change rate into the constant.

7. The state determination method for an endoscope pipe line according to claim 5,
wherein, in the calculation step, the change rate is calculated based on time-division data obtained by time-dividing the physical quantity data for each time.

8. The state determination method for an endoscope pipe line according to claim 7,
wherein, in the calculation step, the change rate is calculated by performing linear approximation of the time-division data.

9. The state determination method for an endoscope pipe line according to claim 7,
wherein, in the calculation step, the change rate is calculated based on a slope between two points included in the time-division data.

10. The state determination method for an endoscope pipe line according to claim 7,
wherein, in the calculation step, the change rate is calculated by performing linear approximation based on a residual of the time-division data.

11. The state determination method for an endoscope pipe line according to claim 7,
wherein, in the calculation step, the change rate is calculated by performing linear approximation in which a sum of squares of residuals of the time-division data is minimized.

12. The state determination method for an endoscope pipe line according to claim 5, further comprising:
an outlier exclusion step of specifying an outlier included in the physical quantity data based on the physical quantity data after the conversion processing is performed, to exclude the outlier from the physical quantity data.

13. The state determination method for an endoscope pipe line according to claim 5, further comprising:
a variation determination step of determining a degree of a variation in the physical quantity data based on the physical quantity data after the conversion processing is performed.

14. The state determination method for an endoscope pipe line according to claim 5,
wherein, in the determination step, the determination is performed by comparing the change rate that is converted into the constant by the conversion processing with a determination threshold value indicating whether the endoscope pipe line is open or blocked.

15. The state determination method for an endoscope pipe line according to claim 1,
wherein the determination period is a period after a preset exclusion period has elapsed from the stop of the supply of the fluid.

16. A state determination device for an endoscope pipe line, comprising:
a supply pipe line that is connected to an endoscope pipe line and that supplies a pressurized fluid into the endoscope pipe line;
a physical quantity detection sensor that detects a physical quantity of the fluid; and
a processor,
wherein the processor
acquires a change rate, which is a change amount per unit time of the physical quantity of the fluid within a determination period after the supply of the fluid is stopped, based on the physical quantity of the fluid detected by the physical quantity detection sensor, and
determines a state of the endoscope pipe line based on the calculated change rate.

17. The state determination device for an endoscope pipe line according to claim 16,
wherein the physical quantity is a pressure or a flow rate of the fluid.

18. The state determination device for an endoscope pipe line according to claim 16,
wherein the supply of the fluid is stopped after the endoscope pipe line is filled with the fluid.

19. The state determination device for an endoscope pipe line according to claim 16,
wherein the processor
detects physical quantity data indicating a physical quantity of the fluid corresponding to each of a plurality of time points within the determination period, and
calculates the change rate based on the detected physical quantity data.

20. The state determination device for an endoscope pipe line according to claim 19,
wherein the processor performs conversion processing of converting the change rate into a constant.

21. An endoscope washing and disinfection device comprising:
the state determination device for an endoscope pipe line according to claim 16.
